(19) United States
(12) Patent Application Publication
(10) Pub. No.: US 2026/0069244 A1
(43) Pub. Date: Mar. 12, 2026

(54) MEDICAL DEVICE AND METHOD FOR MANUFACTURING MEDICAL DEVICE

(71) Applicant: ASAHI INTECC CO., LTD., Seto-shi (JP)

(72) Inventors: Makoto NISHIGISHI, Seto-shi (JP); Yoshiki KATO, Seto-shi (JP); Suzune TSUJI, Seto-shi (JP); Itsuki SUGIWAKA, Seto-shi (JP); Hirotomo SHIMIZU, Seto-shi (JP); Yusuke KIDO, Seto-shi (JP)

(73) Assignee: ASAHI INTECC CO., LTD., Seto-shi (JP)

(21) Appl. No.: 19/382,305

(22) Filed: Nov. 7, 2025

Related U.S. Application Data
(63) Continuation of application No. PCT/JP2023/018776, filed on May 19, 2023.

(51) Int. Cl. A61B 8/00; A61B 90/00

(52) U.S. Cl. CPC A61B 8/445; A61B 90/39; A61B 2090/3966

(57) ABSTRACT

A medical device includes a distal tip, a first tube that has a first lumen, a distal end of the first tube being located closer to a distal end side than a proximal end of the distal tip and on an inner peripheral side of the distal tip, and a second tube that has a second lumen and is in contact with the distal tip.

### CROSS-REFERENCE TO RELATED APPLICATION

The present application claims priority to PCT/JP2023/018776, filed on May 19, 2023, the entire contents of which being incorporated herein by reference.

### TECHNICAL FIELD

The disclosed embodiments relate to a medical device and a method for manufacturing the medical device.

### BACKGROUND ART

Patent Literature 1 discloses a medical device that includes a transducer unit that transmits and receives ultrasonic waves to and from a living tissue, an image lumen that allows the transducer unit and a drive shaft to move, and a guide wire lumen that allows a guide wire to be inserted therethrough. Hereinafter, an ultrasonic image acquisition mechanism including the drive shaft and the transducer unit is also referred to as a “sensor”. By performing a percutaneous procedure using the above medical device, the safety and efficiency of the procedure can be improved.

### CITATION LIST

### Patent Literature

Patent Literature 1: JP 2017-153621 A

### SUMMARY

### Technical Problems

Here, in a medical device having a function of acquiring ultrasonic images, in order to enable operation of a therapeutic device (e.g., a plasma guide wire or a penetration guide wire) under sensor observation, a plurality of lumens is provided by combining a plurality of tubes. In this regard, since the device disclosed in Patent Literature 1 has a configuration in which a second tubular body is joined to a distal end portion of a first tubular body, there is a concern that the second tubular body may fall off in a case where the joint strength between the first tubular body and the second tubular body is not sufficient or depending on the use state of the medical device.

Such an issue is not limited to medical devices in which a sensor is incorporated (that is, a device in which a medical device itself has a function of acquiring ultrasonic images), and is common to medical devices that are configured such that a sensor is insertable and is used in combination with a sensor. In addition, such an issue is common to medical devices inserted into not only the blood vascular system but also various organs in the human body, such as the lymphatic system, biliary system, urinary system, airway system, digestive system, secretory gland, and reproductive organ.

The disclosed embodiments have been made to solve at least a part of the above-described issue and is directed to improving, in a medical device including a plurality of tubes, the joint strength of the plurality of tubes.

### Solutions to Problems

The disclosed embodiments have been made to solve at least a part of the above-described and other issues and can be realized as the following aspects.

(1) According to an aspect of the disclosed embodiments, a medical device is provided. The medical device includes a distal tip, a first tube that has a first lumen, a distal end of the first tube being located closer to a distal end side than a proximal end of the distal tip and on an inner peripheral side of the distal tip, and a second tube that has a second lumen and is in contact with the distal tip.

It should be noted that the disclosed embodiments can be realized in various modes and, for example, can be realized in modes such as a medical device, a medical tube, a catheter, and manufacturing methods thereof.

### BRIEF DESCRIPTION OF DRAWINGS

FIG. 1 is an explanatory view illustrating a configuration of a medical device.

FIG. 2 is an explanatory view illustrating a configuration of the medical device.

FIGS. 3A to 3E are transverse sectional views of a catheter.

FIG. 4 is a diagram illustrating a method of using the catheter.

FIG. 5 is a diagram illustrating a method of using the catheter.

FIG. 6 is an enlarged view of a distal end side of the catheter.

FIGS. 7A and 7B are transverse sectional views of constituent members of the catheter.

FIGS. 8A to 8F are diagrams illustrating a method for manufacturing the catheter.

FIGS. 9A to 9C are diagrams illustrating the method for manufacturing the catheter.

FIG. 10 is an enlarged view of a distal end side of a catheter according to a second embodiment.

FIG. 11 is an enlarged view of a distal end side of a catheter according to a third embodiment.

FIG. 12 is an enlarged view of a distal end side of a catheter according to a fourth embodiment.

FIG. 13 is a transverse sectional view of a catheter according to a fifth embodiment.

### DETAILED DESCRIPTION

### First Embodiment

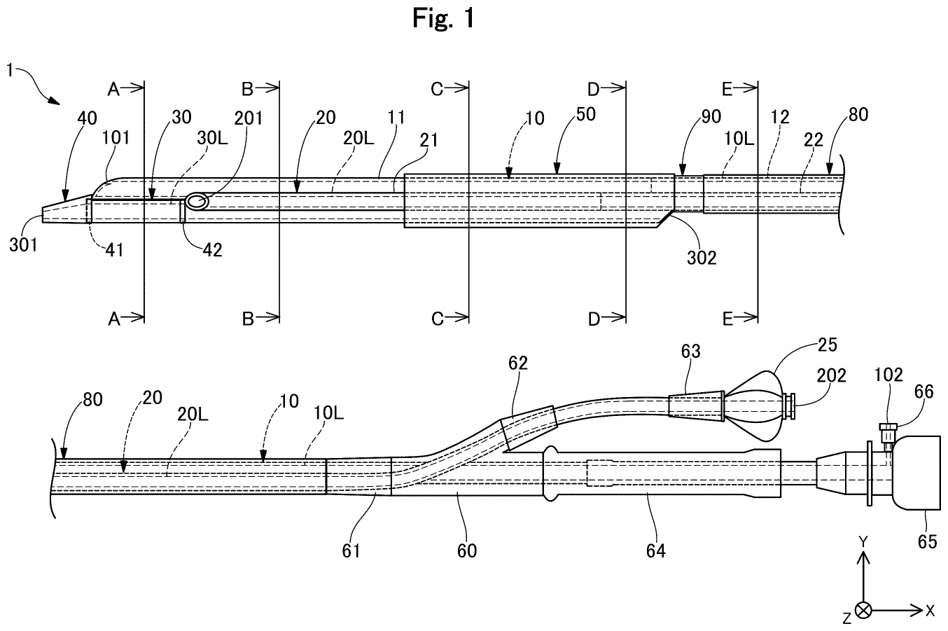

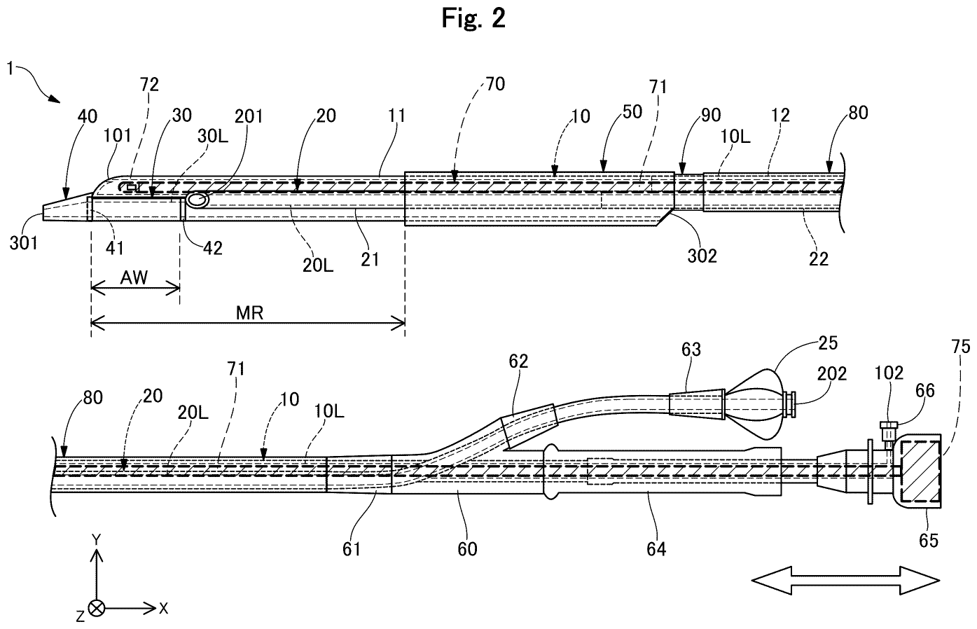

FIGS. 1 and 2 are explanatory views illustrating a configuration of a medical device 1. The medical device 1 according to the present embodiment is a catheter. Hereinafter, the medical device 1 is also referred to as the “catheter 1”. The catheter 1 is used to treat a lesion in a living body lumen such as a CTO occurring in a blood vessel. As illustrated in FIGS. 1 and 2, the catheter 1 includes a sensor tube 10, an OTW (Over The Wire) tube 20, an RX (Rapid Exchange) tube 30, a distal tip 40, a first marker 41, a second marker 42, a first outer tube 50, a branch connector 60, a first reinforcing member 61 to a third reinforcing member 63, a cylindrical member 64, a connector 65, a connector 25, a sensor 70, a second outer tube 80, and a heat-shrinkable tube 90. The sensor tube 10 is also referred to as the “medical device” or “medical tube”. The OTW tube 20 is also referred to as the “medical device” or “medical tube”.

In FIG. 1, the sensor 70 is not illustrated in order to explain the configurations of the tube and the lumen in the tube. In FIG. 2, the sensor 70 incorporated in a sensor lumen 10L in the sensor tube 10 is indicated by a broken line and hatched with oblique lines.

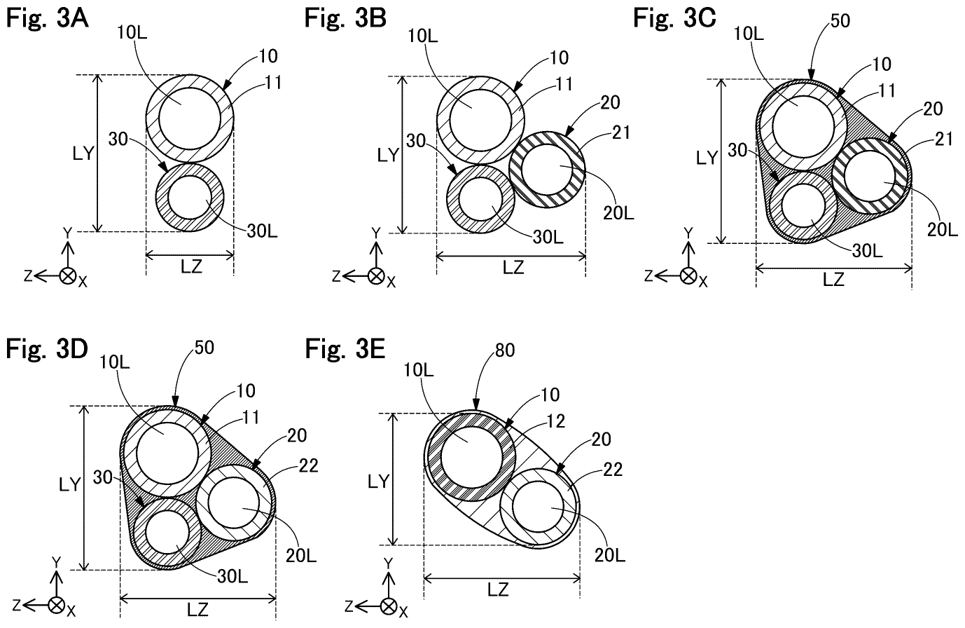

In FIGS. 1 and 2, for convenience of description, the relative ratio of the size of each constituent member includes a portion different from the actual portion. Further, a part of each constituent member includes an exaggerated portion. FIGS. 1 and 2 illustrate the XYZ axes orthogonal to each other. The X-axis corresponds to the longitudinal direction of the catheter 1, the Y-axis corresponds to the height direction of the catheter 1, and the Z-axis corresponds to the width direction of the catheter 1. The left side (−X-axis direction) of FIGS. 1 and 2 is referred to as the “distal end side” of the catheter 1 and each constituent member, and the right side (+X-axis direction) of FIGS. 1 and 2 is referred to as the “proximal end side” of the catheter 1 and each constituent member. In addition, among both ends of the catheter 1 and each constituent member in the longitudinal direction (X-axis direction), one end located on the distal end side is referred to as the “distal end”, and the other end located on the proximal end side is referred to as the “proximal end”. The distal end and the vicinity thereof are referred to as the “distal end portion”, and the proximal end and the vicinity thereof are referred to as the “proximal end portion”. The distal end side is inserted into a living body, and the proximal end side is operated by a professional such as a doctor. These points are common to FIGS. 3A to 3E and subsequent drawings.

FIGS. 3A to 3E are transverse sectional views of the catheter 1. FIG. 3A illustrates a transverse section of the catheter 1 taken along the line A-A in FIG. 1. FIG. 3B illustrates a transverse section of the catheter 1 taken along the line B-B in FIG. 1. FIG. 3C illustrates a transverse section of the catheter 1 taken along the line C-C in FIG. 1. FIG. 3D illustrates a transverse section of the catheter 1 taken along the line D-D in FIG. 1. FIG. 3E illustrates a transverse section of the catheter 1 taken along the line E-E in FIG. 1. Hereinafter, the configuration of the catheter 1 will be described with reference to FIGS. 1 to 3E.

The sensor tube 10 is a hollow cylindrical member (tubular body) having an elongated outer shape. The sensor tube 10 extends linearly along the longitudinal direction (X-axis direction) of the catheter 1 in parallel with the OTW tube 20 and the RX tube 30. The sensor lumen 10L (broken line) for accommodating the sensor 70 is formed inside the sensor tube 10. The sensor lumen 10L is a lumen for the sensor 70.

In the longitudinal direction of the catheter 1, the distal end of the sensor tube 10 is located at the same position as the distal end of the RX tube 30 or slightly closer to the proximal end side than the distal end of the RX tube 30. A distal end opening 101 communicating between the distal end of the sensor lumen 10L and the outside is formed at the distal end of the sensor tube 10. The distal end opening 101 is a fluid discharge port for bringing the inside of the sensor lumen 10L into a wet state. The proximal end of the sensor tube 10 is located closer to the proximal end side than the proximal end of the OTW lumen 20L and the proximal end of the RX tube 30 in the longitudinal direction of the catheter 1. The first reinforcing member 61, the branch connector 60, the cylindrical member 64, and the connector 65 are attached on the proximal end side of the sensor tube 10 from the distal end side toward the proximal end side. Details will be described below. A fluid supply portion 66 is attached to the connector 65, and a proximal end opening 102 communicating between the proximal end of the sensor lumen 10L and the outside is formed in the fluid supply portion 66. The proximal end opening 102 is a fluid supply port to the sensor lumen 10L.

As illustrated in FIG. 1, the sensor tube 10 includes a distal end side tube 11 provided on the distal end side and a proximal end side tube 12 provided closer to the proximal end side than the distal end side tube 11. Both the distal end side tube 11 and the proximal end side tube 12 are hollow cylindrical members (tubular bodies) having an elongated outer shape. The distal end side tube 11 and the proximal end side tube 12 are connected to each other inside the first outer tube 50 in the longitudinal direction. Specifically, both the distal end side tube 11 and the proximal end side tube 12 form a part of the sensor lumen 10L.

The OTW tube 20 is a hollow cylindrical member (tubular body) having an elongated outer shape. On the distal end side of the branch connector 60, the OTW tube 20 extends linearly along the longitudinal direction of the catheter 1 in parallel with the sensor tube 10 and the RX tube 30. An OTW lumen 20L (broken line) for accommodating a therapeutic device (for example, a plasma guide wire or a penetration guide wire) is formed inside the OTW tube 20. The OTW lumen 20L has no proximal end opening in a portion that is located in the living body lumen when the catheter 1 is used. The OTW lumen 20L is an over-the-wire (OTW) type lumen.

The distal end of the OTW tube 20 is located closer to the proximal end side than the distal end of the sensor tube 10 and the distal end of the RX tube 30 in the longitudinal direction of the catheter 1. A distal end opening 201 communicating between the distal end of the OTW lumen 20L and the outside is formed at the distal end of the OTW tube 20. The distal end opening 201 is a device projection port for projecting a therapeutic device toward a living tissue. Since the distal end portion of the OTW tube 20 is obliquely cut, the distal end opening 201 is oriented in a direction intersecting with the longitudinal direction of the catheter 1. Thus, when the catheter 1 is used, the therapeutic device can easily reach the living tissue present around the catheter 1. The proximal end of the OTW tube 20 is located closer to the distal end side than the proximal end of the sensor tube 10 and closer to the proximal end side than the proximal end of the RX tube 30 in the longitudinal direction of the catheter 1. The first reinforcing member 61, the branch connector 60, the second reinforcing member 62, the third reinforcing member 63, and the connector 25 are attached on the proximal end side of the OTW tube 20 from the distal end side toward the proximal end side. Details will be described below. A proximal end opening 202 communicating between the proximal end of the OTW lumen 20L and the outside is formed in the connector 25. The proximal end opening 202 is a device insertion port for inserting a therapeutic device into the OTW lumen 20L.

As illustrated in FIG. 1, the OTW tube 20 includes a distal end side tube 21 provided on the distal end side and a proximal end side tube 22 provided closer to the proximal end side than the distal end side tube 21. Both the distal end side tube 21 and the proximal end side tube 22 are hollow cylindrical members (tubular bodies) having an elongated outer shape. The distal end side tube 21 and the proximal end side tube 22 are connected to each other inside the first outer tube 50 in the longitudinal direction. That is, both the distal end side tube 21 and the proximal end side tube 22 form a part of the OTW lumen 20L.

The RX tube 30 is a hollow cylindrical member (tubular body) having an elongated outer shape. The RX tube 30 extends linearly along the longitudinal direction of the catheter 1 in parallel with the sensor tube 10 and the OTW tube 20. An RX lumen 30L (broken line) for accommodating a work hose wire is formed inside the RX tube 30.

The distal end of the RX tube 30 is located at the same position as the distal end of the sensor tube 10 or slightly closer to the distal end side than the distal end of the sensor tube 10 in the longitudinal direction of the catheter 1. A hollow distal tip 40 is joined to the distal end portion of the RX tube 30. A distal end opening 301 communicating between the distal end of the RX lumen 30L and the outside is formed at the distal end of the distal tip 40. The distal end opening 301 is a wire insertion port for inserting a work hose wire into the RX lumen 30L. The proximal end of the RX tube 30 is located closer to the distal end side than the proximal end of the sensor tube 10 and the proximal end of the OTW tube 20 in the longitudinal direction of the catheter 1. A proximal end opening 302 communicating between the proximal end of the RX lumen 30L and the outside is formed at the proximal end of the RX tube 30. The proximal end opening 302 is a wire drawing port for drawing out the work hose wire to the outside. Since the proximal end of the RX tube 30 is obliquely cut, the proximal end opening 302 is oriented in a direction intersecting with the longitudinal direction of the catheter 1. Thus, when the catheter 1 is used, the work hose wire can be easily drawn out from the proximal end opening 302.

The distal tip 40 has radiopacity and is a cylindrical member in which the outer diameter expands from the distal end side toward the proximal end side. The distal tip 40 is joined to the distal end portion of the RX tube 30 and is thus located at the distal end of the catheter 1 and advances in the living body lumen prior to the other members. The inner cavity of the distal tip 40 communicates with the RX lumen 30L of the RX tube 30 and, as described above, the distal end opening 301 communicating between the distal end of the RX lumen 30L and the outside is formed at the distal end of the distal tip 40.

The first marker 41 and the second marker 42 are annular members having radiopacity. The first marker 41 is provided such that the proximal end of the first marker 41 and the proximal end of the distal tip 40 are at the same position in the longitudinal direction of the catheter 1. The first marker 41 is embedded between the outer peripheral surface of the RX tube 30 and the inner peripheral surface of the distal tip 40. The second marker 42 is provided such that the proximal end of the second marker 42 and the distal end of the distal end opening 201 are at the same position in the longitudinal direction of the catheter 1. The second marker 42 is joined to the outer peripheral surface of the RX tube 30. For joining the first marker 41 and the second marker 42, for example, joining between resins by thermal melting or joining with an adhesive such as an epoxy-based adhesive can be employed. Further, the second marker 42 may be colored to be directly visible by the professional. As described above, by arranging the first marker 41 and the second marker 42 on the RX tube 30, it is possible to prevent the first marker 41 and the second marker 42 from interfering with sensing (acquisition of image information) by the sensor 70. The first marker 41 is also simply referred to as the “marker”.

As illustrated in FIG. 3A, in the transverse section taken along the line A-A, the sensor tube 10 (specifically, the distal end side tube 11) and the RX tube 30 are provided, and the outer peripheral surfaces thereof are joined to each other. As illustrated in FIG. 3B, in the transverse section taken along the line B-B, the sensor tube 10 (specifically, the distal end side tube 11), the OTW tube 20 (specifically, the distal end side tube 21), and the RX tube 30 are provided, and the outer peripheral surfaces thereof are joined to each other. As illustrated in FIG. 3C, in the transverse section taken along the line C-C, the sensor tube 10 (specifically, the distal end side tube 11), the OTW tube 20 (specifically, the distal end side tube 21), and the RX tube 30 are covered with the first outer tube 50. Specifically, the outer peripheral surfaces of the three tubes 10, 20, and 30 are covered with the first outer tube 50 formed by melting, and thus the three tubes 10, 20, and 30 are integrally fixed. As illustrated in FIG. 3D, in the transverse section taken along the line D-D, the sensor tube 10 (specifically, the distal end side tube 11), the OTW tube 20 (specifically, the proximal end side tube 22), and the RX tube 30 are covered with the first outer tube 50, as in FIG. 3C. As illustrated in FIG. 3E, in the transverse section taken along the line E-E, the sensor tube 10 (specifically, the proximal end side tube 12) and the OTW tube 20 (specifically, the proximal end side tube 22) are covered with the second outer tube 80. Specifically, the outer peripheral surfaces of the two tubes 10 and 20 are covered with the second outer tube 80 formed by melting, and thus the two tubes 10 and 20 are integrally fixed.

In the A-A transverse section and the B-B transverse section, the sensor tube 10, the OTW tube 20, and the RX tube 30 may be joined together by using any joining material such as an epoxy-based adhesive, or may be welded by heat. In the A-A transverse section, the B-B transverse section, the C-C transverse section, and the D-D transverse section, a height LY of the catheter 1 is greater than a width LZ of the catheter 1. On the other hand, in the E-E transverse section, the height LY of the catheter 1 is smaller than the width LZ of the catheter 1. As illustrated in FIGS. 3A to 3E, the size relationship among the outer diameters of the three tubes 10, 20, and 30 is the outer diameter of the sensor tube 10 >the outer diameter of the OTW tube 20 >the outer diameter of the RX tube 30. In addition, the size relationship among the inner diameters (lumens) of the three tubes 10, 20, and 30 is the inner diameter of the sensor lumen 10L >the inner diameter of the OTW lumen 20L >the inner diameter of the RX lumen 30L. However, the size relationship among the outer diameters and the inner diameters is merely an example, and may be arbitrarily changed.

The A-A transverse section and the B-B transverse section, in other words, the outer shape of the catheter 1 closer to the distal end side than the first outer tube 50 is the shape along the contour of the two tubes 10 and 30 (or the three tubes 10, 20, and 30) arranged adjacent to each other, and a constricted portion (recess portion) is formed in the adjacent portion of each tube. The C-C transverse section and the D-D transverse section, in other words, the outer shape of the catheter 1 in the portion covered with the first outer tube 50 is a triangular shape with round corners (a round-corner triangular shape). The E-E transverse section, in other words, the outer shape of the catheter 1 in the portion covered with the second outer tube 80 is elliptical.

With reference back to FIG. 1, the description will be continued. The three tubes 10, 20, and 30 (specifically, the sensor tube 10, the OTW tube 20, and the RX tube 30) are fixed by the three tubes 90, 50, and 80.

The heat-shrinkable tube 90 is provided between the first outer tube 50 and the second outer tube 80 in the longitudinal direction of the catheter 1. The heat-shrinkable tube 90 covers the sensor tube 10 (specifically, a part of the proximal end side tube 12 on the distal end side) and the OTW tube 20 (specifically, a part of the proximal end side tube 22 on the distal end side) to bundle the two tubes 10 and 20. The heat-shrinkable tube 90 does not cover the RX tube 30, and the RX tube 30 is provided along the outer peripheral surface of the heat-shrinkable tube 90 in a state where the outer peripheral surface of the heat-shrinkable tube 90 and the outer peripheral surface of the RX tube 30 are in contact with each other. The distal end of the heat-shrinkable tube 90 is located closer to the proximal end side than the distal end of the first outer tube 50 and closer to the distal end side than the proximal end opening 302. That is, the distal end portion of the heat-shrinkable tube 90 is covered with the first outer tube 50. The proximal end of the heat-shrinkable tube 90 is located closer to the proximal end side than the distal end of the second outer tube 80 and closer to the distal end side than the first reinforcing member 61. That is, the proximal end portion of the heat-shrinkable tube 90 is covered with the second outer tube 80. In other words, an intermediate portion of the heat-shrinkable tube 90 in the longitudinal direction of the catheter 1 is not covered with the first outer tube 50 or the second outer tube 80.

The first outer tube 50 is located closer to the distal end side than the heat-shrinkable tube 90 in the longitudinal direction of the catheter 1. The first outer tube 50 is provided in a section which is closer to the proximal end side than the distal end opening 201 and in which the three tubes 10, 20, and 30 extend side by side. In the example of FIG. 1, the distal end of the first outer tube 50 is located near the center between the distal end opening 201 and the proximal end opening 302. The proximal end of the first outer tube 50 is located near the proximal end of the proximal end opening 302. As described above, the first outer tube 50 may be provided at a position away from the distal end opening 201 toward the proximal end side. This can prevent the first outer tube 50 from interfering with sensing (acquisition of image information) by the sensor 70 inserted into the sensor lumen 10L. The first outer tube 50 covers and fixes the distal end portion of the heat-shrinkable tube 90, the sensor tube 10 (specifically, a part of the distal end side tube 11 on the proximal end side) exposed from the distal end of the heat-shrinkable tube 90, the OTW tube 20 (specifically, a part of the distal end side tube 21 on the proximal end side and a part of the proximal end side tube 22 on the distal end side) exposed from the distal end of the heat-shrinkable tube 90, and the RX tube 30. As illustrated in FIGS. 3C and 3D, the first outer tube 50 has a triangular outer shape with round corners and has a thick portion formed by melting along the outer peripheral surfaces of the three tubes 10, 20, and 30.

The second outer tube 80 is located closer to the proximal end side than the heat-shrinkable tube 90 in the longitudinal direction of the catheter 1. The second outer tube 80 is provided in a section which is closer to the proximal end side than the proximal end opening 302 and in which the two tubes 10 and 20 extend side by side. In the example of FIG. 1, the distal end of the second outer tube 80 is located at a portion slightly away from the proximal end opening 302 toward the proximal end side. The proximal end of the second outer tube 80 is located inside the first reinforcing member 61. The second outer tube 80 covers and fixes the proximal end portion of the heat-shrinkable tube 90, the sensor tube 10 exposed from the proximal end of the heat-shrinkable tube 90 (specifically, a part of the proximal end side tube 12 on the proximal end side), and the OTW tube 20 exposed from the proximal end of the heat-shrinkable tube 90 (specifically, a part of the proximal end side tube 22). As illustrated in FIG. 3E, the second outer tube 80 has an elliptical outer shape and has a thick portion formed by melting along the outer peripheral surfaces of the two tubes 10 and 20.

The branch connector 60 is a member having a bifurcated inner cavity and is provided on the proximal end side of the catheter 1. The OTW tube 20 is inserted into one inner cavity of the branch connector 60. The sensor tube 10 is inserted into the other inner cavity of the branch connector 60. The first reinforcing member 61 is a hollow cylindrical member provided closer to the distal end side than the branch connector 60. The first reinforcing member 61 covers the outer periphery of the second outer tube 80 in which the sensor tube 10 and the OTW tube 20 are bundled, thereby reinforcing the distal end side of the branch connector 60.

The second reinforcing member 62 is a hollow cylindrical member provided closer to the proximal end side than one branch of the branch connector 60. The second reinforcing member 62 covers the outer periphery of the OTW tube 20 inserted into the branch connector 60, thereby reinforcing the proximal end side of the branch connector 60. The third reinforcing member 63 is a hollow cylindrical member provided closer to the distal end side than the connector 25. The third reinforcing member 63 covers the outer periphery of the OTW tube 20 inserted into the connector 25, thereby reinforcing the distal end side of the connector 25. The connector 25 is a member joined to the proximal end portion of the OTW tube 20. The connector 25 includes a pair of blade portions to be gripped by the professional. The proximal end opening 202 (device insertion port) communicating between the proximal end of the OTW lumen 20L and the outside is formed at the proximal end of the connector 25.

The cylindrical member 64 is a hollow cylindrical member provided closer to the proximal end side than the other branch of the branch connector 60. The cylindrical member 64 covers the outer periphery of the sensor tube 10 inserted into the branch connector 60, thereby reinforcing the proximal end side of the branch connector 60. The connector 65 is a member joined to the proximal end portion of the sensor tube 10. A housing for accommodating a connection terminal 75 of the sensor 70 is provided on the proximal end side of the connector 65. The fluid supply portion 66, in which the proximal end opening 102 communicating between the proximal end of the sensor lumen 10L and the outside is formed, is provided on the outer peripheral surface of the connector 65.

The sensor 70 (FIG. 2) is an imaging sensor that acquires image information. As illustrated in FIG. 2, the sensor 70 includes a main body part 71, a probe 72, and a connection terminal 75. The main body part 71 is an elongated member extending along the longitudinal direction of the catheter 1. A driving cable (coaxial line) that electrically connects the probe 72 and the connection terminal 75 is incorporated inside the main body part 71. The probe 72 includes an ultrasonic probe (also referred to as an ultrasonic vibrator, a piezoelectric body, an ultrasonic transmission/reception element, or an ultrasonic element) that transmits an ultrasonic wave toward a living tissue and receives the ultrasonic wave propagated through the living tissue and reflected. The probe 72 is also referred to as an imaging core or a transducer. The connection terminal 75 is a terminal that electrically connects the sensor 70 to a console terminal provided outside. The connection terminal 75 is provided at the proximal end of the main body part 71 and is accommodated in the housing of the connector 65.

The sensor 70 is electrically connected to an external console terminal via the connection terminal 75, receives the power supplied from the console terminal, and outputs a detection signal by the probe 72 to the console terminal. Thus, the console terminal can display the image information based on the detection signal of the probe 72. As illustrated in FIG. 2, the sensor 70 is fixed to the connector 65. Further, as indicated by the white arrow in FIG. 2, the professional grips the connector 65 and slides the connector 65 in the front-rear direction (the direction of the white arrow) and thus can move the position of the probe 72 of the sensor 70 within a range MR from the distal end of the sensor lumen 10L to the distal end of the first outer tube 50, in other words, within the predetermined range MR including the distal end opening 201. Hereinafter, the range MR is also referred to as the “movable range MR”. Further, a portion of the catheter 1 which is particularly suitable for sensing (acquisition of image information) by the sensor 70 is also referred to as an “acoustic window AW”. As illustrated in FIG. 2, the acoustic window AW is a section between the first marker 41 and the second marker 42 in the catheter 1.

The distal end side tube 11 of the sensor tube 10, the distal end side tube 21 of the OTW tube 20, and the RX tube 30 can be formed of a flexible material, for example, a thermoplastic resin such as a polyethylene resin, a polypropylene resin, or polyurethane, polyvinyl chloride, ethylene-vinyl acetate copolymers, cross-linked ethylene-vinyl acetate copolymers, polyamide elastomer, polyolefin elastomer, polyurethane elastomer, silicone rubber, or latex rubber. The distal end side tube 11 of the sensor tube 10, the distal end side tube 21 of the OTW tube 20, and the RX tube 30 may be formed of the same material or different materials.

The proximal end side tube 12 of the sensor tube 10 and the proximal end side tube 22 of the OTW tube 20 can be formed of, for example, a resin having high rigidity such as a nylon resin, a polyester resin, or a PEEK resin. The melting points of the proximal end side tube 12 of the sensor tube 10 and the proximal end side tube 22 of the OTW tube 20 are higher than the melting points of the above-described tubes 11, 21, and 30. The proximal end side tube 12 of the sensor tube 10 and the proximal end side tube 22 of the OTW tube 20 may be formed of the same material or different materials.

In the catheter 1 according to the present embodiment, by providing the section in which a part of the RX tube 30 having flexibility on the proximal end side is overlapped with the proximal end side tube 12 and the proximal end side tube 22 having high rigidity (FIG. 1), gradual changes in rigidity of the catheter 1 is achieved (a rigidity gap of the catheter 1 is reduced). As a result, kinking of the catheter 1 can be suppressed. Further, one or more of the distal end side tube 11 and the proximal end side tube 12 of the sensor tube 10, the distal end side tube 21 and the proximal end side tube 22 of the OTW tube 20, and the RX tube 30 may have a multilayer structure in which tubes made of different materials are stacked.

The distal tip 40, the first marker 41, and the second marker 42 can be formed of a resin material or a metal material having radiopacity. For example, when a radiopaque resin material is used, it can be formed by mixing a radiopaque material such as bismuth trioxide, tungsten, or barium sulfate with a polyamide resin, a polyolefin resin, a polyester resin, a polyurethane resin, a silicone resin, or a fluororesin. For example, when a radiopaque metal material is used, it can be formed of gold, platinum, tungsten, or an alloy containing these elements (for example, a platinum-nickel alloy). The distal tip 40, the first marker 41, and the second marker 42 may be formed of the same material or different materials.

The branch connector 60, the first reinforcing member 61 to the third reinforcing member 63, the cylindrical member 64, the connector 65, and the connector 25 can be formed of a known resin material. The branch connector 60, the first reinforcing member 61 to the third reinforcing member 63, the cylindrical member 64, the connector 65, and the connector 25 may be formed of the same material or different materials.

The heat-shrinkable tube 90 is formed of a nylon-based elastomer resin (for example, polyamide elastomer) having thermoplasticity. The heat-shrinkable tube 90 has a property of shrinking without melting when heated. In addition, the heat-shrinkable tube 90 has improved adhesiveness (property of easily sticking to another substance) at the time of heating compared to that at the time of non-heating. The heat-shrinkable tube 90 may be formed of polyolefin, FEP (Fluorinated Ethylene Propylene), or silicone.

The first outer tube 50 and the second outer tube 80 are formed of a nylon-based elastomer resin having thermoplasticity. Unlike the heat-shrinkable tube 90, the first outer tube 50 and the second outer tube 80 have a property of melting when heated. In the example according to the present embodiment, a resin having lower shore hardness than the second outer tube 80 is used for the first outer tube 50. However, the first outer tube 50 and the second outer tube 80 may be formed of the same material or may be formed of different materials.

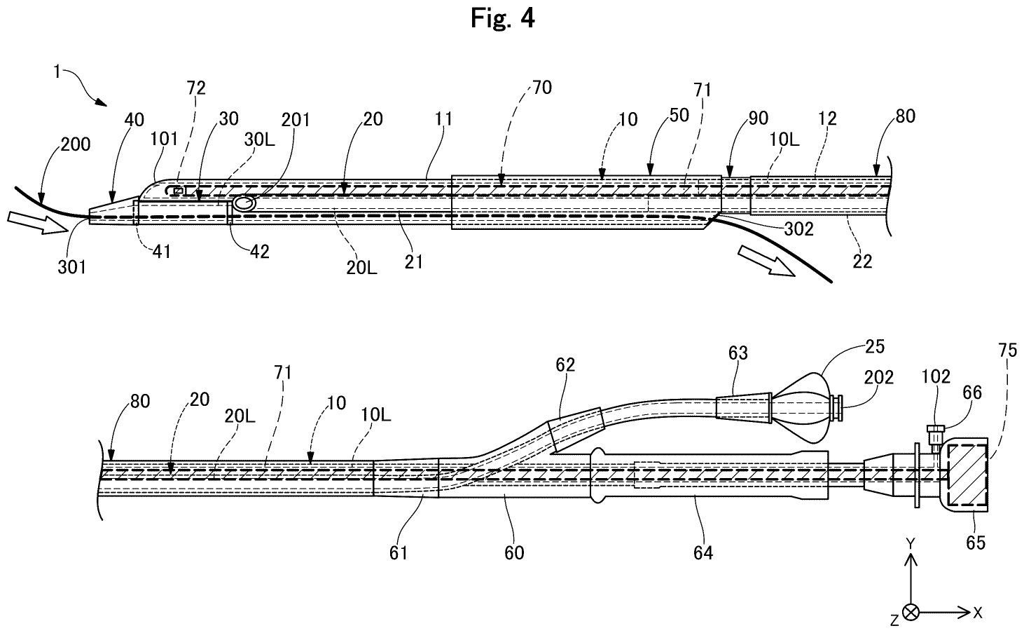

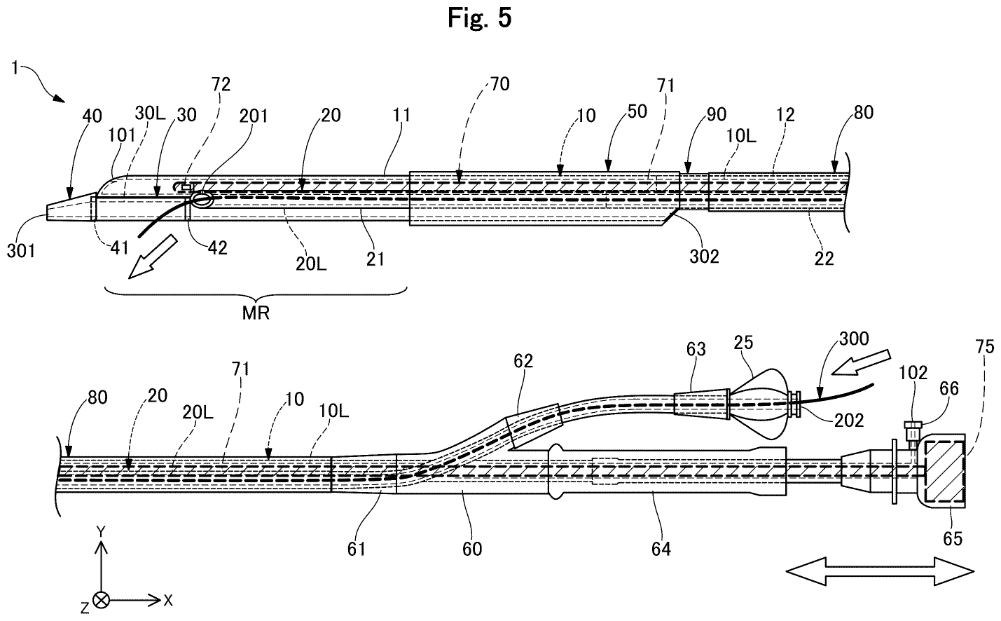

FIGS. 4 and 5 are diagrams illustrating a method of using the catheter 1. In the following steps a1 to a6, the case of recanalization of a CTO (lesion) generated in a blood vessel by the forward approach will be exemplified. However, the catheter 1 may be used in an inverse approach and may be used for operation other than recanalization of a CTO.

(a1) The professional inserts the work hose wire 200 into the blood vessel and delivers the distal end portion of the work hose wire 200 to the vicinity of the CTO. (a2) The professional inserts the proximal end portion of the work hose wire 200 from the distal end opening 301 of the catheter 1, passes it through the RX lumen 30L, and draws it out from the proximal end opening 302 of the catheter 1 (FIG. 4). (a3) The professional pushes the catheter 1 into the blood vessel along the work hose wire 200 and delivers the distal end portion of the catheter 1 to the vicinity of the CTO. In the step a3, the catheter 1 may be delivered to the vicinity of the CTO by passing the catheter 1 through a guiding catheter inserted into the blood vessel in advance along the work hose wire 200. (a4) While adjusting the position of the probe 72 of the sensor 70 within the movable range MR by gripping the connector 65 and sliding the connector 65 in the front-rear direction (FIG. 5: the direction of the white arrow), the professional checks the image displayed on the console terminal and thus matches the positions and orientations of the CTO and the distal end opening 201. The position means the position in the extending direction of the blood vessel, and the orientation means the orientation in the circumferential direction of the inner wall of the blood vessel. (a5) The professional inserts the distal end portion of a therapeutic device 300 from the proximal end opening 202 of the catheter 1, inserts the distal end portion into the OTW lumen 20L, and protrudes the distal end portion from the distal end opening 201 of the catheter 1 (FIG. 5). (a6) The professional treats the CTO using the therapeutic device 300 while adjusting the position of the probe 72 of the sensor 70 within the movable range MR as necessary and checking the image displayed on the console terminal. As described above, any device such as a plasma guide wire or a penetration guide wire can be used as the therapeutic device 300.

The sensor tube 10, the OTW tube 20, and the RX tube 30 are also collectively referred to as a “shaft”. The sensor tube 10 (the distal end side tube 11 and the proximal end side tube 12) corresponds to a “first tube”. The RX tube 30 corresponds to a “second tube”. The OTW tube 20 (the distal end side tube 21 and the proximal end side tube 22) corresponds to a “third tube”. The sensor lumen 10L corresponds to a “first lumen”, the RX lumen 30L corresponds to a “second lumen”, and the OTW lumen 20L corresponds to a “third lumen”. The distal end opening 201 corresponds to a “distal end opening”. According to the present embodiment, “the same” and “equal” are not limited to a case of an exact match, and have a meaning of allowing a difference due to a manufacturing error or the like. In addition, “constant” is synonymous with “substantially constant”, and means substantially constant while allowing a deviation due to a manufacturing error or the like.

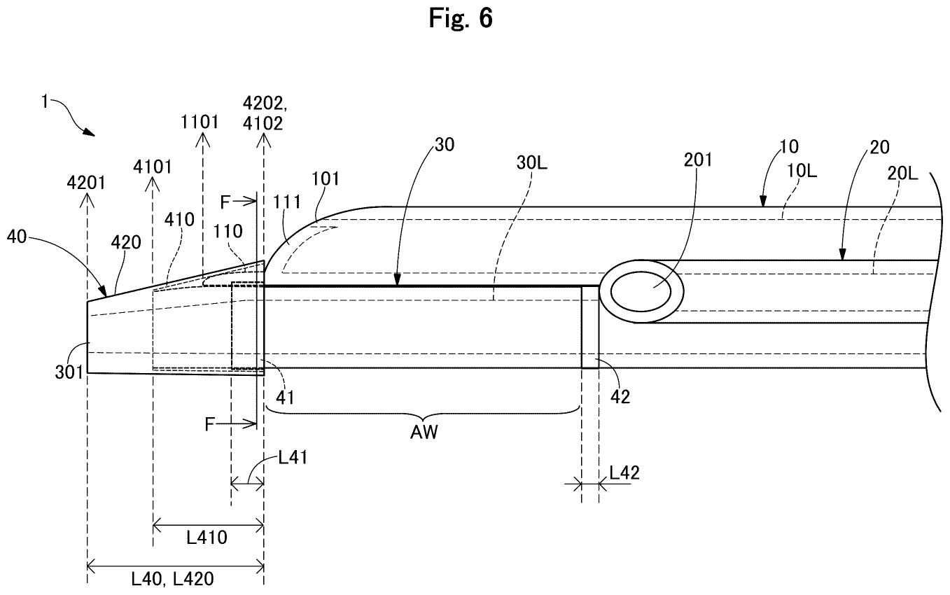

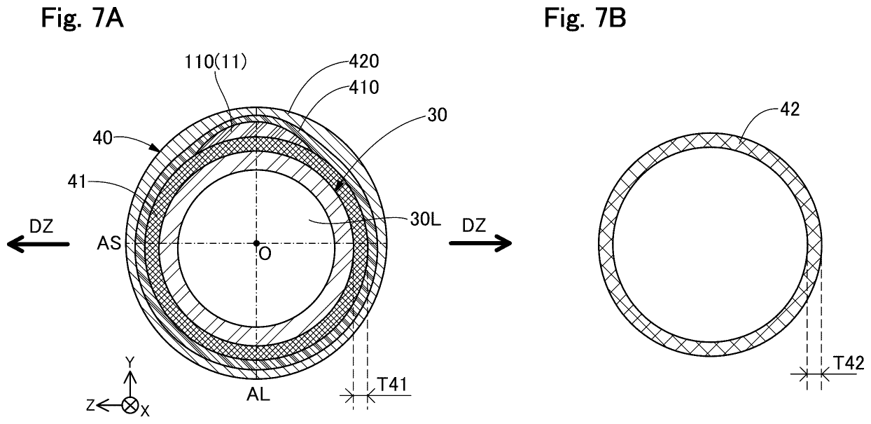

FIG. 6 is an enlarged view of the distal end side of the catheter 1. FIGS. 7A and 7B are transverse sectional views of constituent members of the catheter 1. FIG. 7A illustrates a transverse section of the catheter 1 taken along the line F-F in FIG. 6, in other words, a transverse section of the catheter 1 at the proximal end portion of the distal tip 40. FIG. 7B illustrates a transverse section of the second marker 42. The distal tip 40, the first marker 41, and the second marker 42 will be described in detail with reference to FIGS. 6, 7A, and 7B.

As illustrated in FIG. 6, the distal tip 40 has a two layer structure including a first layer 410 and a second layer 420. The first layer 410 (inner layer) covers, e.g., encapsulates, and integrally fixes the distal end portion of the RX tube 30 (second tube) and a distal end portion 110 of the sensor tube 10 (first tube). The second layer 420 (outer layer) covers the first layer 410. A distal end 4101 of the first layer 410 is located closer to the proximal end side than a distal end 4201 of the second layer 420. A proximal end 4102 of the first layer 410 is located at the same position as a proximal end 4202 of the second layer 420. The distal end 4201 of the second layer 420 corresponds to the “distal end of the distal tip”, and the proximal end 4202 of the second layer 420 corresponds to the “proximal end of the distal tip”.

A length L40 of the distal tip 40 in the longitudinal direction of the catheter 1 is equal to a length L420 of the second layer 420 (the length L40=L420). Further, the length L420 of the second layer 420 is longer than a length L410 of the first layer 410 (the length L420<L410). Both the first layer 410 and the second layer 420 have radiopacity. The first layer 410 and the second layer 420 can be formed of the radiopaque resin material or the radiopaque metal material described above as the material of the distal tip 40.

As illustrated in FIG. 7A, in the transverse section of the catheter 1 at the proximal end portion of the distal tip 40, the RX tube 30 is provided on the innermost side. The first marker 41 covers the RX tube 30 in a state where the inner peripheral surface of the first marker 41 is in contact with the outer peripheral surface of the RX tube 30. The distal end portion 110 extending along the outer peripheral surface of the RX tube 30 is provided through the first marker 41 on a part of the outer peripheral surface of the first marker 41 (in the illustrated example, a part of the outer peripheral surface in the +Y direction). As illustrated in FIGS. 6 and 7A, the distal end portion 110 is a thick portion having a crescent transverse sectional shape formed by melting a part of the distal end side of the sensor tube 10 that originally has a hollow cylindrical sectional shape. In particular, this thick portion is a result of melting and crushing to deform the distal end portion 110. As illustrated in FIG. 7A, there is no lumen inside the distal end portion 110. The first layer 410 covers the distal end portion 110 and the first marker 41 in a state where the inner peripheral surface of the first layer 410 is in contact with the outer peripheral surface of the distal end portion 110 and the outer peripheral surface of the first marker 41. The second layer 420 covers the first layer 410 in a state where the inner peripheral surface of the second layer 420 is in contact with the outer peripheral surface of the first layer 410.

As illustrated in FIGS. 6 and 7A, the RX tube 30 (second tube) is in contact with the distal tip 40 through the first marker 41 in the portion where the first marker 41 is provided, and is in direct contact with the distal tip 40 on the distal end side of the first marker 41. Further, a distal end 1101 of the sensor tube 10 (first tube) is located closer to the distal end side than the proximal end 4202 of the distal tip and on the inner peripheral side of the distal tip 40. In the sensor tube 10, a raised portion 111 in which the wall of the tube is raised is formed closer to the proximal end side than the distal end portion 110. The diameter of the sensor lumen 10L is reduced on the inner side of the raised portion 111, and a distal end opening 101 allowing a fluid to flow between the inside and the outside of the RX lumen 30L is formed at the end portion of the raised portion 111 (in the illustrated example, the end portion on the side far from the RX tube 30). The diameter of the distal end opening 101 is smaller than the diameter of the sensor 70, and the sensor 70 cannot be inserted through the distal end opening 101.

As illustrated in FIG. 7A, the transverse sectional shape of the catheter 1 at the proximal end portion of the distal tip 40 is 360-degree symmetry. Here, “the transverse sectional shape of the catheter 1 is 360-degree symmetry” means at least one of the following: the contour of the outer periphery of the distal tip 40 (specifically, the second layer 420) in the transverse section is 360-degree symmetry; and the configuration of each member of the catheter 1 in the transverse section is 360-degree symmetry. It should be noted that “360-degree symmetry” means that a symmetrical shape does not appear without one rotation. For this reason, the configuration in which all the members of the catheter 1 have circular transverse sections and these members are concentrically laminated is not 360-degree symmetry (excluded from the concept of 360-degree symmetry, but has infinite symmetry). In other words, the transverse sectional shape of the catheter 1 at the proximal end portion of the distal tip 40 is rotationally asymmetric. Here, “rotationally asymmetric” means that the cross-sectional profile lacks n-fold rotational symmetry for any integer n>1, such that a rotation of less than 360-degrees about its central axis does not result in an identical profile. The reason why the catheter 1 according to the present embodiment has a transverse sectional shape of 360-degree symmetry at the proximal end portion of the distal tip 40 is that the outer peripheral contour shape and the thickness of the first layer 410 and the outer peripheral contour shape and the thickness of the second layer 420 become non-uniform as the distal end portion 110 of the sensor tube 10 is included in the laminated structure.

The outer peripheral contour of the distal tip 40 (specifically, the second layer 420) in the transverse section illustrated in FIG. 7A has a substantially elliptical shape. FIG. 7A illustrates a center O of the ellipse, a long axis AL of the ellipse passing through the center O, and a short axis AS of the ellipse passing through the center O. As illustrated in the drawing, the long axis AL of the ellipse intersects with the sensor tube 10 (first tube), while the short axis AS of the ellipse does not intersect with the sensor tube 10.

The first marker 41 is an annular member having radiopacity and is provided between the RX tube 30 and the first layer 410 (in other words, on the outer peripheral side of the RX tube 30 and on the inner peripheral side of the first layer 410), as illustrated in FIG. 7A. In the longitudinal direction of the catheter 1, the first marker 41 is provided in at least a partial section from the distal end 1101 of the sensor tube 10 (first tube) to the proximal end 4202 of the distal tip. In the example of FIG. 6, the first marker 41 is provided such that the distal end of the first marker 41 is located near the center of the distal end portion 110 and the proximal end of the first marker 41 is located at the proximal end 4202 of the distal tip. Thus, the radiopaque members are provided in three layers (the first marker 41, the first layer 410, and the second layer 420) at the position where the first marker 41 is provided.

The second marker 42 is an annular member having radiopacity and is provided on the outer peripheral side of the RX tube 30 as illustrated in FIG. 6. In the longitudinal direction of the catheter 1, the second marker 42 is provided at a position closer to the proximal end side than the first marker 41 and away from the first marker 41. In the example of FIG. 6, the second marker 42 is provided such that the proximal end of the second marker 42 is located at the distal end of the distal end opening 201 (distal end opening) of the OTW lumen 20L (third lumen). Accordingly, by referring to the position of the second marker 42 on the X-ray image, the professional can recognize the distal end position of the distal end opening 201 from which the therapeutic device protrudes.

As illustrated in FIG. 6, a length L41 of the first marker 41 in the longitudinal direction of the catheter 1 is longer than a length L42 of the second marker 42 (the length L41>L42). On the other hand, as illustrated in FIGS. 7A and 7B, a thickness T41 of the first marker 41 and a thickness T42 of the second marker 42 are the same (the thickness T41=T42). When the first marker 41 and the second marker 42 do not have constant lengths (for example, when the shape viewed from the side is a trapezoidal shape or a triangular shape), the lengths of the longest portions are used as the lengths L41 and L42. Similarly, when the first marker 41 and the second marker 42 have non-uniform thicknesses, the thicknesses of the thickest portions are used as the thicknesses T41 and T42.

The first marker 41 and the second marker 42 may have a shape different from the annular shape. For example, the first marker 41 and the second marker 42 may have a shape obtained by cutting a circular ring at an arbitrary angle, or a linear shape. For example, the first marker 41 and the second marker 42 may have a coil shape in which a wire is spirally wound. The first marker 41 and the second marker 42 may be provided on a tube (for example, the sensor tube 10 or the OTW tube 20) different from the RX tube 30. The first marker 41 and the second marker 42 may be provided on the same tube as described above, or may be provided on different tubes.

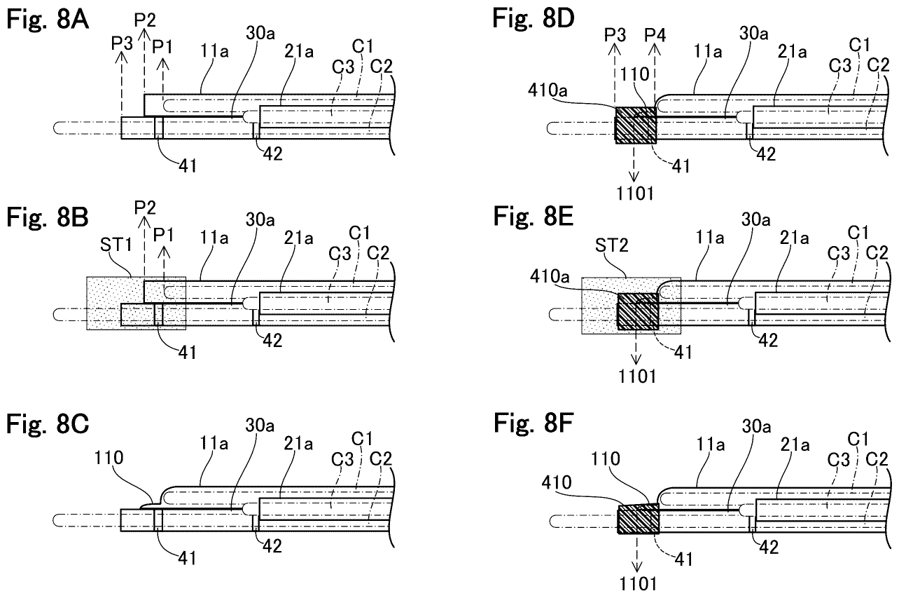

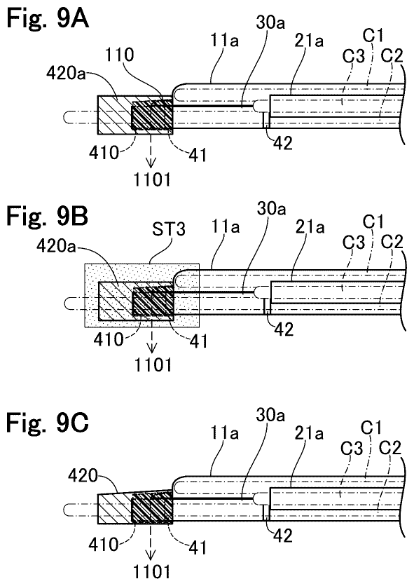

FIGS. 8A to 8F and 9A to 9C are diagrams illustrating a method for manufacturing the catheter 1. In FIGS. 8A to 8F and 9A to 9C, a first hollow cylindrical member and a second hollow cylindrical member are hatched with different types of oblique lines, and the heat-shrinkable tube is hatched with dots. In FIGS. 8A to 8F and 9A to 9C, “a” is added to the end of the reference numeral of a member before a change whose shape is changed by heating or the like in the manufacturing process. First to third heat-shrinkable tubes ST1 to ST3 appearing in the following description are tubular bodies formed of a nylon-based elastomer resin, polyolefin, FEP, silicone, or the like, having thermoplasticity and have a property of shrinking without melting when heated. Further, first and second hollow cylindrical members 410a and 420a are tubular bodies formed of polyurethane resins containing tungsten and, unlike the first to third heat-shrinkable tubes ST1 to ST3, the first and second hollow cylindrical members 410a and 420a have a property of melting when heated. Various materials described above as the material of the distal tip 40 may be used as the first and second hollow cylindrical members 410a and 420a. The first hollow cylindrical member is also simply referred to as the “hollow cylindrical member”.

FIG. 8A illustrates a placement step. As illustrated in FIG. 8A, the operator prepares a distal end side tube 11a (first tube) into which a first cored bar C1 is inserted, an RX tube 30a (second tube) into which a second cored bar C2 is inserted, and a distal end side tube 21a (third tube) into which a third cored bar C3 is inserted. Then, the operator arranges the respective members such that a distal end P1 of the first cored bar C1 is located closer to the proximal end side than a distal end P2 of the distal end side tube 11a and the distal end P2 of the distal end side tube 11a is located closer to the proximal end side than a distal end P3 of the RX tube 30a. In the illustrated example, the distance from the distal end P3 to the distal end P1 is longer than the distance from the distal end P2 to the distal end P1. As illustrated in the figure, the first marker 41 and the second marker 42 are previously provided in the RX tube 30a. The operator arranges the distal end side tube 21a such that the distal end of the distal end side tube 21a is located closer to the proximal end side than the distal end P1 of the first cored bar C1. In the manufacturing method described with reference to FIGS. 8A to 8F and 9A to 9C, the distal end side tube 21a (third tube) does not need to be used and may be omitted.

FIG. 8B illustrates a state before heating in a distal end portion formation step. As illustrated in FIG. 8B, the operator covers at least the range from the distal end P2 of the distal end side tube 11a (first tube) to the distal end P1 of the first cored bar C1 with the first heat-shrinkable tube ST1. In the illustrated example, the first heat-shrinkable tube ST1 covers the range from the distal end P3 of the RX tube 30a (second tube) to the distal end P1 of the first cored bar C1. Then, the operator heats at least the range from the distal end P2 to the distal end P1 at a first temperature. The first temperature is a temperature at which the first heat-shrinkable tube ST1 heat-shrinks and the distal end side tube 11a melts. The first heat-shrinkable tube is also simply referred to as the “heat-shrinkable tube”.

FIG. 8C illustrates a state after heating in the distal end portion formation step. After the heating, the operator removes the first heat-shrinkable tube ST1. Accordingly, as illustrated in FIG. 8C, a portion of the distal end side tube 11a into which the first cored bar C1 is not inserted (i.e., a portion from the distal end P2 to the distal end P1) is melted and crushed along with the heat shrinkage of the first heat-shrinkable tube ST1 so that the distal end portion 110 is formed, which extends along the outer peripheral surface of the RX tube 30a. As illustrated in the drawing, the lumen in the distal end side tube 11a is occluded on the distal end side of the distal end side tube 11a (first tube), and the distal end portion 110 is formed on the distal end side of the occluded end, i.e., the distal end portion is solid. Although the distal end opening 101 (FIG. 6) is formed in the raised portion closer to the proximal end side than the distal end portion 110 after the first cored bar C1 is removed, the formation of the distal end opening 101 may be omitted.

FIG. 8D illustrates a state in which the first hollow cylindrical member 410a is provided in a first layer formation step. As illustrated in FIG. 8D, the operator covers the distal end portion 110 of the distal end side tube 11a (first tube) and the RX tube 30a (second tube) with the first hollow cylindrical member 410a. In the illustrated example, the first hollow cylindrical member 410a covers the entire portion from the distal end P3 of the RX tube 30a to a proximal end P4 of the distal end portion 110 of the distal end side tube 11a.

FIG. 8E illustrates a state in which the second heat-shrinkable tube ST2 is provided in the first layer formation step. As illustrated in FIG. 8E, the operator covers the first hollow cylindrical member 410a with the second heat-shrinkable tube ST2. In the illustrated example, the second heat-shrinkable tube ST2 covers the entire first hollow cylindrical member 410a from the distal end of the first hollow cylindrical member 410a to the proximal end of the first hollow cylindrical member 410a. Then, the operator heats at least the range in which the first hollow cylindrical member 410a is present at a second temperature. The second temperature is a temperature at which the second heat-shrinkable tube ST2 heat-shrinks and the first hollow cylindrical member 410a melts.

FIG. 8F illustrates a state after heating in the first layer formation step. After heating, the operator removes the second heat-shrinkable tube ST2. Then, as illustrated in FIG. 8F, the first hollow cylindrical member 410a is melted and crushed along with the heat shrinkage of the second heat-shrinkable tube ST2 so that the first layer 410 is formed, which extends along the distal end portion 110 and the distal end portion of the RX tube 30a. On the side surface illustrated in FIG. 8F, the first layer 410 has a shape in which the outer edge on the side where the distal end portion 110 is present is inclined as compared with the outer edge on the opposite side (in other words, the transverse section of 360-degree symmetry as described in FIG. 7A), and fixes the distal end portion 110 of the distal end side tube 11a (first tube) and the RX tube 30a (second tube).

FIG. 9A illustrates a state in which the second hollow cylindrical member 420a is provided in a second layer formation step. As illustrated in FIG. 9A, the operator covers the first layer 410 formed by the first hollow cylindrical member 410a with the second hollow cylindrical member 420a. In the illustrated example, the second hollow cylindrical member 420a covers the entire first layer 410 (the first hollow cylindrical member 410a) in a state where the distal end is located closer to the distal end side than the distal end of the first layer 410 (the first hollow cylindrical member 410a) and the proximal end is located at the same position as the proximal end of the first layer 410 (the first hollow cylindrical member 410a).

FIG. 9B illustrates a state in which the third heat-shrinkable tube ST3 is provided in the second layer formation step. As illustrated in FIG. 9B, the operator covers the second hollow cylindrical member 420a with the third heat-shrinkable tube ST3. In the illustrated example, the third heat-shrinkable tube ST3 covers the entire second hollow cylindrical member 420a from the distal end of the second hollow cylindrical member 420a to the proximal end of the second hollow cylindrical member 420a. Then, the operator heats at least the range in which the second hollow cylindrical member 420a is present at a third temperature. The third temperature is a temperature at which the third heat-shrinkable tube ST3 heat-shrinks and the second hollow cylindrical member 420a melts.

FIG. 9C illustrates a distal tip formation step. After the heating, the operator removes the third heat-shrinkable tube ST3. Accordingly, as illustrated in FIG. 9C, the second hollow cylindrical member 420a is melted and welded to the first layer 410 (the first hollow cylindrical member 410a) along with the heat shrinkage of the third heat-shrinkable tube ST3 so that the second layer 420 is formed, which extends along the first layer 410 (the first hollow cylindrical member 410a). On the side surface illustrated in FIG. 9C, the second layer 420 has a shape in which the outer edge on the side where the distal end portion 110 is present is inclined as compared with the outer edge on the opposite side (in other words, the transverse section of 360-degree symmetry as described in FIG. 7A), and fixes the first layer 410 (the first hollow cylindrical member 410a). Then, the operator cuts the distal end side of the second layer 420 formed by the second hollow cylindrical member 420a to have a predetermined dimension required for the distal tip 40, and then tapers the distal end side of the second layer 420. In tapering processing, for example, by heating the distal end portion of the second layer 420 (the second hollow cylindrical member 420a), the distal end portion can be formed into a tapered shape with round corners. Thus, as described in FIG. 6, the distal tip 40 having a two layer structure including the first layer 410 and the second layer 420 can be formed.

As described above, in the catheter 1 according to the first embodiment, as illustrated in FIG. 6, the distal end 1101 of the distal end side tube 11 (first tube) is located closer to the distal end side than the proximal end 4202 of the distal tip and on the inner peripheral side of the distal tip 40, and the RX tube 30 (second tube) is in contact with the distal tip 40 so that both the distal end side tube 11 (first tube) and the RX tube 30 can be fixed by using the distal tip 40. As a result, the joint strength of the first and second tubes can be improved as compared with the case where the first and second tubes are joined without using the distal tip 40.

In addition, in the catheter 1 according to the first embodiment, as illustrated in FIG. 6, the first marker 41 is provided in at least a partial section along the longitudinal direction of the catheter 1 (medical device) from the distal end 1101 of the distal end side tube 11 (first tube) to the proximal end 4202 of the distal tip. Therefore, even when the distal tip 40 is not formed of a radiopaque material, the position of the distal tip 40 can be grasped by the first marker 41. In addition, in a case where the distal tip 40 is formed of a radiopaque material as in the above-described embodiment, the members formed of radiopaque materials have a multilayer structure so that an image under X-ray imaging can be denser.

In addition, in the catheter 1 according to the first embodiment, the second marker 42 having radiopacity is provided at a position closer to the proximal end side than the first marker 41 and away from the first marker 41. For this reason, since the two markers (the first marker 41 and the second marker 42) are provided at positions separated from each other in the longitudinal direction of the catheter 1 (medical device), the visibility of the catheter 1 under X-ray imaging can be improved. Further, in the catheter 1 according to the first embodiment, since the second marker 42 is located near the distal end opening 201 of the OTW lumen 20L (third lumen), the position of the distal end opening 201 of the third lumen can be grasped by the second marker 42.

Furthermore, in the catheter 1 according to the first embodiment, since the distal tip 40 includes the first layer 410 and the second layer 420 covering the first layer 410 as illustrated in FIG. 6, the visibility of the distal tip 40 under X-ray imaging can be improved in the portion where the first layer 410 and the second layer 420 overlap each other. Further, as illustrated in the above embodiment (FIG. 6), when the first marker 41 is provided in the section from the distal end 1101 of the distal end side tube 11 (first tube) to the proximal end 4202 of the distal tip, the visibility of the distal tip under X-ray imaging can be further improved in the portion where the first layer 410, the second layer 420, and the first marker 41 overlap each other.

Furthermore, in the catheter 1 according to the first embodiment, as illustrated in FIG. 7A, the distal tip 40 has a transverse sectional shape that is 360-degree symmetry at the proximal end portion, and therefore, in a procedure using the catheter 1 (medical device), the catheter 1 can be easily bent in the direction intended by the professional. Specifically, the catheter 1 can be easily bent in specific directions indicated by arrows DZ in FIG. 7A, as compared with a case where all the members of the catheter 1 have a circular transverse section. Further, in the catheter 1 according to the first embodiment, as illustrated in FIG. 7A, the outer periphery of the distal tip 40 has a substantially elliptical shape in which the long axis AL intersects with the distal end side tube 11 (first tube) and the short axis AS does not intersect with the distal end side tube 11 (first tube). Therefore, the catheter 1 (medical device) can be easily bent in the short axis direction DZ of the outer periphery of the distal tip 40.

Furthermore, in the catheter 1 according to the first embodiment, since the length L41 of the first marker 41 is longer than the length L42 of the second marker 42, the image of the first marker 41 can be longer than the image of the second marker 42 on the X-ray image. As a result, the professional can easily distinguish between the first marker 41 and the second marker 42.

Further, in the method for manufacturing the catheter 1 according to the first embodiment, by the placement step illustrated in FIG. 8A, the distal end portion 110 having a shape suitable for fixation using the distal tip 40 (specifically, the first hollow cylindrical member 410a and the second hollow cylindrical member 420a) can be formed on the distal end side of the distal end side tube 11a (first tube). Further, in the placement step illustrated in FIG. 8A, the distal end position P1 of the first cored bar C1 and the distal end position P2 of the distal end side tube 11a (first tube) are adjusted so that the length of the distal end portion 110 can be controlled.

Furthermore, in the method for manufacturing the catheter 1 according to the first embodiment, in the first layer formation step illustrated in FIGS. 8D to 8F, the distal end portion 110 of the distal end side tube 11a (first tube) and the RX tube 30a (second tube) are nipped and fixed by using the second cored bar C2 and the first hollow cylindrical member 410a, and thus the distal end side tube 11a and the RX tube 30a can be firmly fixed. Further, the distal tip 40 having a two layer structure can be formed by the second layer formation step illustrated in FIGS. 9A and 9B.

### Second Embodiment

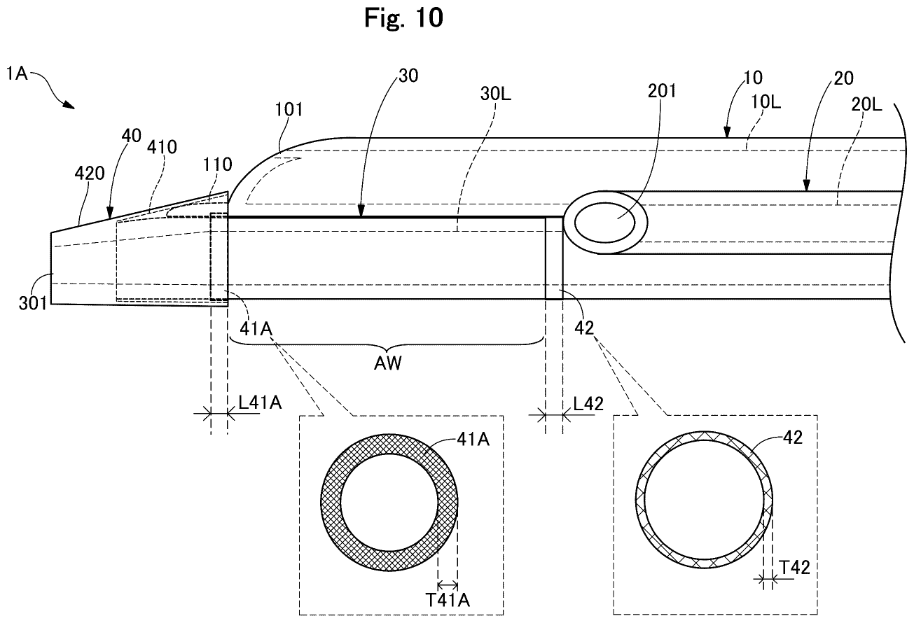

FIG. 10 is an enlarged view of the distal end side of a catheter 1A according to a second embodiment. The catheter 1A according to the second embodiment includes a first marker 41A instead of the first marker 41 in the configuration described in the first embodiment. In the lower section of FIG. 10, the transverse sectional view of the first marker 41A and the transverse sectional view of the second marker 42 are illustrated in balloons.

A length L41A and a thickness T41A of the first marker 41A are different from those in the first embodiment. To be specific, the length L41A of the first marker 41A in the longitudinal direction of the catheter 1A is the same as the length L42 of the second marker 42. That is, the length L40>L41A=L42. As illustrated inside the balloons, the thickness T41A of the first marker 41 is thicker than the thickness T42 of the second marker 42 (the thicknesses T41A>T42).

As described above, the configurations of the first marker 41A and the second marker 42 can be variously changed. In the example of FIG. 10, the length L41A of the first marker 41A is equal to that of the second marker 42, and the thickness T41A of the first marker 41A is thicker than that of the second marker 42. However, the configuration may be such that the length L41A of the first marker 41A is longer than that of the second marker 42 and the thickness T41A of the first marker 41A is thicker than that of the second marker 42 (L41A>L42, T41A>T42). Further, the configuration may be such that the length L41A of the first marker 41A is equal to that of the second marker 42 and the thickness T41A of the first marker 41A is also equal to that of the second marker 42 (L41A=L42, T41A=T42). In this case, when the first marker 41A and the second marker 42 are manufactured, the type or the mixing ratio of the radiopaque material to be mixed with the resin material may be changed so that the visibility of the first marker 41A in the X-ray image becomes higher than that of the second marker 42.

In the catheter 1A according to the second embodiment as described above, too, the same effects as those of the first embodiment described above can be achieved. Further, in the catheter 1A (medical device) according to the second embodiment, since the thickness T41A of the first marker 41A is thicker than the thickness T42 of the second marker 42, the image of the first marker 41A can be darker than the image of the second marker 42 on the X-ray image. As a result, the professional can easily distinguish between the first marker 41A and the second marker 42.

### Third Embodiment

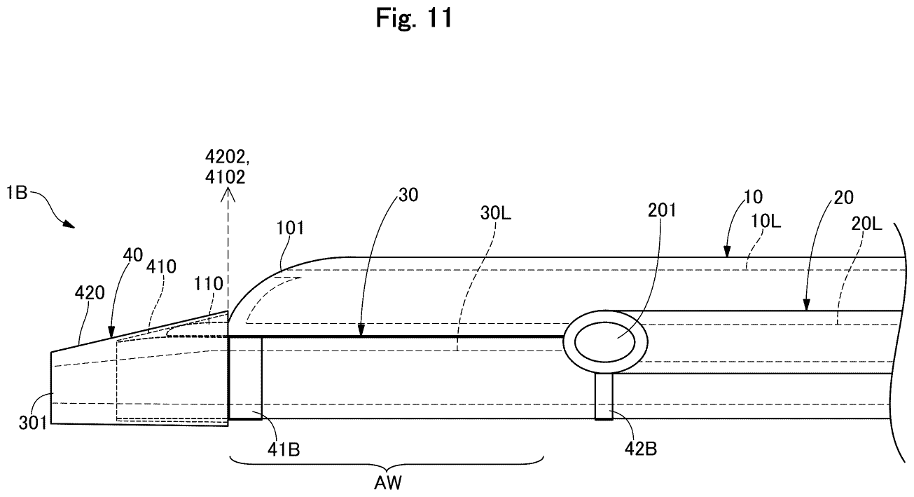

FIG. 11 is an enlarged view of the distal end side of a catheter 1B according to a third embodiment. The catheter 1B according to the third embodiment includes a first marker 41B instead of the first marker 41 and a second marker 42B instead of the second marker 42 in the configuration described in the first embodiment.

The first marker 41B is provided at a position not overlapping with the distal tip 40. In the illustrated example, the first marker 41B is provided at a position where the distal end of the first marker 41B abuts the proximal end 4202 of the distal tip 40. In other words, a distal end surface of the first marker 41B is in contact with a proximal end surface of the distal tip 40. The second marker 42B is provided such that the center of the second marker 42B is located at the center of the distal end opening 201 (distal end opening) of the OTW lumen 20L (third lumen) in the longitudinal direction of the catheter 1B.

As described above, the configurations of the first marker 41B and the second marker 42B can be variously changed. The first marker 41B does not need to overlap the distal tip 40 while being in contact with the distal tip 40, and the second marker 42B may be provided at any position near the distal end opening 201. Also, in the catheter 1B according to the third embodiment as described above, the same effects as those of the first embodiment described above can be achieved.

### Fourth Embodiment

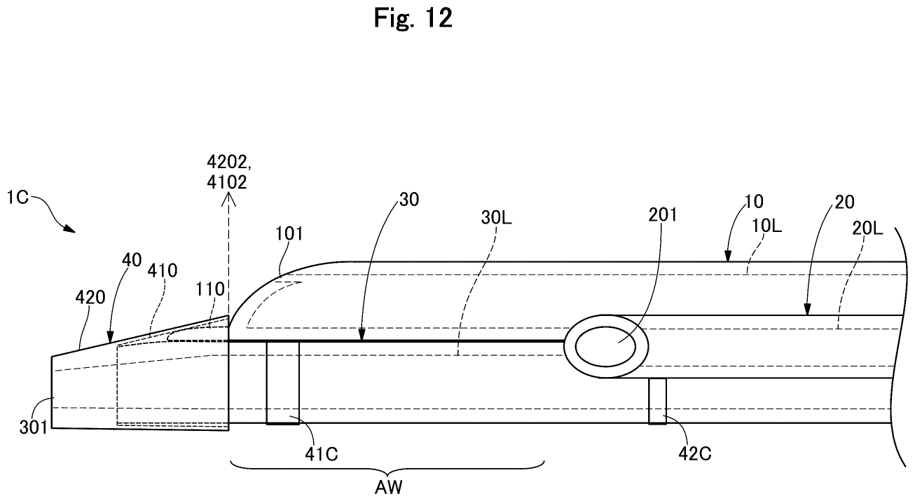

FIG. 12 is an enlarged view of the distal end side of a catheter 1C according to a fourth embodiment. The catheter 1C according to the fourth embodiment includes a first marker 41C instead of the first marker 41 and a second marker 42C instead of the second marker 42 in the configuration described in the first embodiment. The first marker 41C does not overlap the distal tip 40, and is provided at a position away from the distal tip 40. In addition, the second marker 42C is provided near the distal end opening 201 of the OTW lumen 20L (third lumen) and at a position closer to the proximal end side than the distal end opening 201. As described above, the configurations of the first marker 41C and the second marker 42C can be variously changed. The first marker 41C does not need to be in contact with or overlap the distal tip 40, and the second marker 42C may be provided at any position near the distal end opening 201. The catheter 1C according to the fourth embodiment described above can also achieve the same effects as those of the first embodiment described above.

### Fifth Embodiment

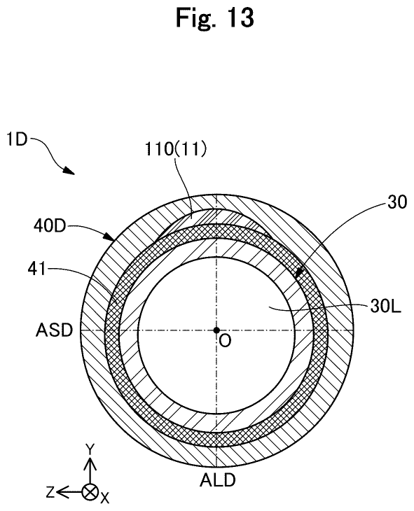

FIG. 13 is a transverse sectional view of a catheter 1D according to a fifth embodiment. FIG. 13 illustrates a transverse section of the catheter 1D taken along the line F-F in FIG. 6, in other words, a transverse section of the catheter 1D at the proximal end portion of the distal tip 40D. The catheter 1D according to the fifth embodiment includes a distal tip 40D instead of the distal tip 40 in the configuration described in the first embodiment.

The distal tip 40D includes a single layer. In the transverse section illustrated in FIG. 13, the contour of the outer periphery of the distal tip 40D is a perfect circle. Thus, the axis ASD=ALD passes through the center O of the circle. The contour of the outer peripheral of the distal tip 40D in the transverse section of FIG. 13 is not 360-degree symmetry. On the other hand, the configuration of each member of the catheter 1D in the transverse section of FIG. 13 is 360-degree symmetry. This is due to the presence of the distal end portion 110.

As described above, the configuration of the distal tip 40D can be variously changed, and the distal tip 40D may be formed of a single layer. In this case, the second layer formation step described in FIGS. 9A to 9C may be omitted. Further, the distal tip 40D may have a layer configuration of three or more layers. In this case, after the second layer formation step described in FIGS. 9A to 9C, a third layer may be stacked on the second layer 420 by using a third hollow cylindrical member. Also, the distal tip 40D may have a transverse sectional shape other than an elliptical shape. Although a perfect circle is illustrated in FIG. 13, the shape is not limited thereto. Also, in the catheter 1D according to the fifth embodiment described above, the same effects as those of the first embodiment described above can be achieved.

### Modification of Present Embodiment

The disclosed embodiments are not limited to the above-described embodiments, and can be implemented in various modes without departing from the gist of the disclosed embodiments, and for example, the following modifications are also possible.

### [Modification 1]

In the first to fifth embodiments, examples of the configurations of the catheters 1 and 1A to 1D have been described. However, the configurations of the catheters 1 and 1A to 1D can be variously changed.

For example, at least one of the sensor tube 10 and the OTW tube 20 may omit the distal end side tubes 11 and 21 and the proximal end side tubes 12 and 22 and may be configured by one tubular body (tubular body) from the distal end to the proximal end. At least one of the tubes 10, 20, and 30 may be configured by three or more tubular bodies (tubular bodies) provided along the longitudinal direction of the catheter 1.

For example, the outer peripheral surfaces of the first outer tube 50, the heat-shrinkable tube 90, and the second outer tube 80 or the outer peripheral surface of the catheter 1 including these may be coated with a hydrophilic resin or a hydrophobic resin. For example, the sensor 70 is incorporated in the sensor lumen 10L of the sensor tube 10 and is not removable from the catheter 1. However, the sensor 70 may be configured to be removable from the catheter 1. That is, the catheter 1 may omit the sensor 70 as a constituent element.

For example, at least one of the first marker 41 and the second marker 42 may be omitted. For example, the shapes of the first marker 41 and the second marker 42 can be arbitrarily changed. The first marker 41 and the second marker 42 may have a shape different from the annular shape (for example, a shape obtained by cutting an annular ring at an arbitrary angle, a linear shape, or a coil shape obtained by spirally winding a wire). For example, the first marker 41 and the second marker 42 may be provided on a tube (the sensor tube 10 or the OTW tube 20) different from the RX tube 30. The first marker 41 and the second marker 42 may be provided on the same tube as described above, or may be provided on different tubes.

For example, at least any of the first outer tube 50, the second outer tube 80, and the heat-shrinkable tube 90 may be omitted. For example, in the example according to the above-described embodiment, each of the tubes 50, 80, and 90 is formed of one layer, but at least any of the tubes 50, 80, and 90 may be formed of two or more layers. For example, in the example according to the embodiment described above, the transverse sectional shape of the portion of the catheter 1 covered with the first outer tube 50 is a triangular shape with round corners, but may be any shape such as a circular shape or an elliptical shape. For example, in the example according to the embodiment described above, the transverse sectional shape of the portion of the catheter 1 covered with the second outer tube 80 is an elliptical shape, but may be any shape such as a circular shape or a triangular shape with round corners. For example, at least any of the first outer tube 50 and the second outer tube 80 may have an outer shape along the contour of the outer peripheral surface of the tubes 10 and 20 (or the tubes 10, 20, and 30).

For example, the shapes of the branch connector 60, the first reinforcing member 61 to the third reinforcing member 63, the cylindrical member 64, the connector 65, and the connector 25 described above are merely examples, and may be arbitrarily changed. For example, at least a part of the branch connector 60, the first reinforcing member 61, the second reinforcing member 62, and the cylindrical member 64 may be configured as a single member or may be omitted. For example, the third reinforcing member 63 and the connector 25 may be configured as one member. For example, the cylindrical member 64 may include a mechanism (e.g., a scale or a stopper provided for each predetermined length in the longitudinal direction, or a scale or a stopper provided for each predetermined angle in the circumferential direction) that assists adjustment of at least one of the front-back position of the sensor 70 and the orientation of the sensor 70 in the circumferential direction.

For example, the method for manufacturing the catheters 1 and 1A to 1D described in FIGS. 8A to 8F and 9A to 9C is merely an example, and various modifications can be made. For example, pretreatment for processing or separate processing for placing other members which are not described may be further provided between the above-described steps.

### [Modification 2]

The configurations of the catheters 1 and 1A to 1D according to the first to fifth embodiments and the configurations of the catheters 1 and 1A to 1D according to the modification 1 may be appropriately combined. For example, the first and second markers 41 and 42 described in any one of the second to fourth embodiments may be combined with the distal tip 40D described in the fifth embodiment.

### Aspects

(1) According to an aspect of the disclosed embodiments, a medical device is provided. The medical device includes a distal tip, a first tube that has a first lumen, a distal end of the first tube being located closer to a distal end side than a proximal end of the distal tip and on an inner peripheral side of the distal tip, and a second tube that has a second lumen and is in contact with the distal tip.

With this configuration, the distal end of the first tube is located closer to the distal end side than the proximal end of the distal tip and on the inner peripheral side of the distal tip, and the second tube is in contact with the distal tip; therefore, both the first tube and the second tube can be fixed by using the distal tip. As a result, the joint strength of the first and second tubes can be improved as compared with the case where the first and second tubes are joined without using the distal tip.

(2) The medical device according to the above-described aspect may further include a marker that is provided at the second tube and has radiopacity, and the marker may be provided in at least a partial section along a longitudinal direction of the medical device from the distal end of the first tube to the proximal end of the distal tip. With this configuration, the position of the distal tip can be grasped by the marker.

(3) In the medical device according to the above-described aspect, the marker may be a first marker, and the medical device may further include a second marker that has radiopacity and is located at a position closer to a proximal end side than the first marker and away from the first marker. With this configuration, since the two markers (the first marker and the second marker) are provided at the positions separated from each other in the long axis direction of the medical device, it is possible to improve the visibility of the medical device under X-ray imaging (angiographic image).

(4) The medical device according to the above-described aspect may further include a third tube that has a third lumen and a distal end opening communicating between a distal end of the third lumen and outside, the distal end opening being located near the second marker. With this configuration, the position of the distal end opening of the third lumen can be grasped by the second marker.

(5) In the medical device according to the above-described aspect, the distal tip may include a first layer and a second layer covering the first layer, a distal end of the first layer may be located closer to a proximal end side than a distal end of the second layer, and both the first layer and the second layer may have radiopacity. With this configuration, it is possible to improve the visibility of the distal tip under X-ray imaging in the portion where the first layer and the second layer overlap each other. In addition, in this configuration, in a case where the first marker is provided in a section from the distal end of the first tube to the proximal end of the distal tip, it is possible to further improve the visibility of the distal tip under X-ray imaging in the portion where the first layer, the second layer, and the first marker overlap each other.

(6) In the medical device according to the above-described aspect, a shape of a transverse section at a proximal end portion of the distal tip may be symmetric in 360 degrees. With this configuration, the distal tip has a transverse sectional shape that is symmetric in 360 degrees at the proximal end portion, and therefore, in a procedure using the medical device, the medical device can be easily bent in the direction intended by the professional.

(7) In the medical device according to the above-described aspect, in the transverse section, an outer periphery of the distal tip may have a substantially elliptical shape in which a long axis intersects with the first tube and a short axis does not intersect with the first tube. With this configuration, the medical device can be easily bent in the short axis direction of the outer periphery of the distal tip.

(8) According to an aspect of the disclosed embodiments, a method for manufacturing a medical device is provided. The method for manufacturing the medical device includes: placing a first tube into which a first cored bar is inserted and a second tube into which a second cored bar is inserted such that a distal end of the first cored bar is located closer to a proximal end side than a distal end of the first tube and the distal end of the first tube is located closer to the proximal end side than a distal end of the second tube; and covering from the distal end of the first tube to the distal end of the first cored bar with a heat-shrinkable tube and, by using heat shrinkage of the heat-shrinkable tube, on a distal end side of the first tube, forming a distal end portion in which a lumen in the first tube is occluded and which extends along an outer peripheral surface of the second tube on a distal end side of an occluded end. With this manufacturing method, the distal end portion having a shape suitable for fixation using the distal tip can be formed on the distal end side of the first tube. Further, in the placement step, the distal end position of the first cored bar and the distal end position of the first tube are adjusted so that the length of the distal end portion can be controlled.

(9) In the method for manufacturing the medical device according to the above-described aspect, the heat-shrinkable tube may be a first heat-shrinkable tube, and the method may include: after forming the distal end portion, covering the distal end portion and the second tube with a hollow cylindrical member made of a resin; covering the hollow cylindrical member with a second heat-shrinkable tube; and fixing the hollow cylindrical member to the distal end portion of the first tube and the second tube by using heat shrinkage of the second heat-shrinkable tube. With this manufacturing method, since the distal end portion of the first tube and the second tube are nipped and fixed by using the second cored bar and the hollow cylindrical member, the first tube and the second tube can be firmly fixed.

(10) In the method for manufacturing the medical device according to the above-described aspect, the hollow cylindrical member may be a first hollow cylindrical member, and the method may include: covering the first hollow cylindrical member with a second hollow cylindrical member made of a resin; welding the second hollow cylindrical member to fix the second hollow cylindrical member to the first hollow cylindrical member; and tapering a distal end side of the second hollow cylindrical member to form a distal tip. With this manufacturing method, the distal tip having a two layer structure can be formed.

Although the present mode has been described above based on the embodiments and the modifications, the embodiment of the above-described mode is intended to facilitate understanding of the present mode and does not limit the present mode. The present mode can be modified and improved without departing from the gist and the scope of the claims, and the present mode includes equivalents thereof. In addition, when the technical features are not described as essential in the present specification, the technical features can be appropriately deleted.

What is claimed is:

1. A medical device comprising:
a distal tip having a proximal end and an outer periphery;
a first tube that has a first lumen and a distal end, wherein the distal end is distal to the proximal end of the distal tip and within the outer periphery of the distal tip; and
a second tube that has a second lumen and is in contact with the distal tip.

2. The medical device according to claim 1, further comprising:
a marker on the second tube and having radiopacity, wherein
the marker is on at least a partial section along a longitudinal direction of the medical device between the distal end of the first tube and the proximal end of the distal tip.

3. The medical device according to claim 2, wherein
the marker is a first marker, and
the medical device further comprising a second marker having radiopacity and is located at a position proximal to and longitudinally spaced apart from the first marker.

4. The medical device according to claim 3, further comprising:
a third tube that has a third lumen and a distal end opening communicating between a distal end of the third lumen and outside, wherein the second marker indicates a position of the distal end opening.

5. The medical device according to claim 3, wherein a longitudinal length of the first marker is greater than a longitudinal length of the second marker.

6. The medical device according to claim 3, wherein a radial thickness of the first marker is greater than a radial thickness of the second marker.

7. The medical device according to claim 1, wherein
the distal tip includes a first layer and a second layer covering the first layer,
a distal end of the first layer is located proximal to a distal end of the second layer, and
both the first layer and the second layer have radiopacity.

8. The medical device according to claim 5, wherein the first layer of the distal tip encapsulates both the distal end of the first tube and a portion of the second tube.

9. The medical device according to claim 1, wherein
a shape of a transverse section at a proximal end portion of the distal tip is rotational asymmetric.

10. The medical device according to claim 9, wherein,
in the transverse section, an outer periphery of the distal tip has a substantially elliptical shape in which a long axis intersects with the first tube and a short axis does not intersect with the first tube.

11. The medical device according to claim 1, wherein a distal portion of the first tube is deformed to occlude the first lumen and conform to an outer surface of the second tube.

12. The medical device according to claim 1, wherein the first tube is a sensor tube and the first lumen is configured to receive an image sensor.

13. A method for manufacturing a medical device comprising:
arranging a first tube having a first cored bar inserted therein and a second tube having a second cored bar inserted therein such that a distal end of the first cored bar is positioned proximal to a distal end of the first tube and the distal end of the first tube is positioned proximal to a distal end of the second tube;
covering from the distal end of the first tube to the distal end of the first cored bar with a heat-shrinkable tube and, by using heat shrinkage of the heat-shrinkable tube, on a distal end side of the first tube, forming a distal end portion in which a lumen in the first tube is occluded and which extends along an outer peripheral surface of the second tube on a distal end side of an occluded end.

14. The method for manufacturing the medical device according to claim 13, wherein
the heat-shrinkable tube is a first heat-shrinkable tube, and
the method further comprising:
after forming the distal end portion, disposing a hollow cylindrical member made of a resin over the distal end portion and the second tube;
covering the hollow cylindrical member with a second heat-shrinkable tube; and
fixing the hollow cylindrical member to the distal end portion of the first tube and the second tube by using heat shrinkage of the second heat-shrinkable tube.

15. The method for manufacturing the medical device according to claim 14, wherein
the hollow cylindrical member is a first hollow cylindrical member,
the method further comprising:
covering the first hollow cylindrical member with a second hollow cylindrical member made of a resin;
welding the second hollow cylindrical member to fix the second hollow cylindrical member to the first hollow cylindrical member; and
tapering a distal end side of the second hollow cylindrical member to form a distal tip.

16. The method for manufacturing the medical device according to claim 13, further comprising providing a radiopaque marker on the second tube.
